(19) United States
(12) Patent Application Publication
(10) Pub. No.: US 2009/0247842 A1
(43) Pub. Date: Oct. 1, 2009

(54) Interferometric Measuring Instrument for Taking Optical Measurements on Skin Structures

(76) Inventor: Ulrich Kallmann, Tuebingen (DE)

Correspondence Address: KENYON & KENYON LLP, ONE BROADWAY, NEW YORK, NY, 10004, US

(21) Appl. No.: 12/227,052

(22) PCT Filed: Mar. 20, 2007
(86) PCT No.: PCT/EP2007/052603

§ 371 Date: May 15, 2009

(30) Foreign Application Priority Data
May 4, 2006 (DE) 10 2006 020 720.3

(51) Int. Cl. A61B 5/1455

(52) U.S. Cl. 600/316

(57) ABSTRACT

An interferometric measuring instrument for taking optical measurements on skin structures, having a beam splitter for splitting an input beam, emitted by a light source, into an object beam and a reference beam; having an object branch via which the object beam is directed to the skin, and the object beam, scattered back from the skin structures, is returned; having a reference branch via which the reference beam is directed to a reference and returned from it; and having a detector device for picking up the recombined and interfering radiation of the back-scatted object beam and of the returned reference beam and for evaluating the intensity modulation obtained due to the interference. The following measures contribute significantly to a reliable and rapid optical measurement, e.g., of the blood-sugar concentration: In order to scatter back a portion of the object beam directed into the skin, an implant having a front side facing the incident object beam and a back side facing away from it is introduced into the skin, the implant having a refractive index different from the surrounding skin substance.

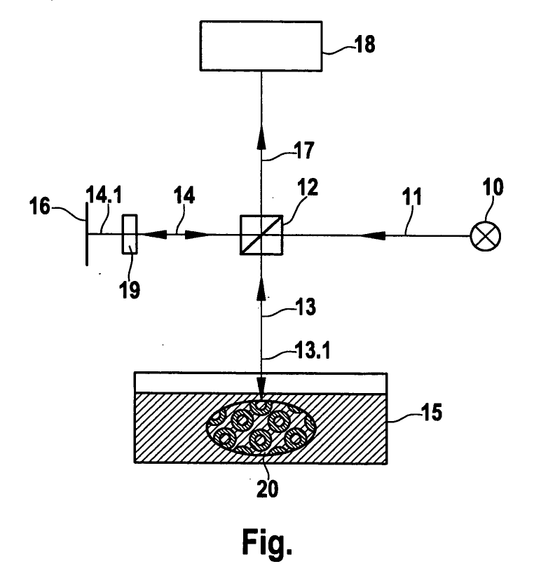

### FIELD OF THE INVENTION

The present invention relates to an interferometric measuring instrument for taking optical measurements on skin structures, having a beam splitter for splitting an input beam, emitted by a light source, into an object beam and a reference beam; having an object branch via which the object beam is directed to the skin, and the object beam, scattered back from the skin structures, is returned; having a reference branch via which the reference beam is directed to a reference and returned from it; and having a detector device for picking up the recombined and interfering radiation of the back-scattered object beam and of the returned reference beam and for evaluating the intensity modulation obtained due to the interference.

### BACKGROUND INFORMATION

An interferometric measuring instrument is specified in R. Kuranov, D. Prough, V. Sapozhnikova, I. Cicenaite, R. Esenaliev, “In vivo application of 2-D lateral scanning mode Optical Coherence Tomography for glucose sensing”, in Proc. SPIE, 6007, 60070K-1 60070K-6 (2005). In this known measuring instrument and this known measuring method, respectively, the blood-sugar concentration is ascertained using optical coherence tomography (OCT) by optically determining the sugar concentration in the tissue fluid of the skin with the aid of the interferometric measuring instrument on the basis of a change in refractive index, the index change being measured indirectly via the depth-resolved measurement of the light scattered back into the optical apparatus. In this case, the measurement is based on the finding that the extent of the scattering is influenced considerably by the refractive index of the tissue fluid; the scattering intensity is a function of the optical refractive-index jump that exists between the tissue fluid and scattering bodies in the skin (e.g., skin cells). A disadvantage of this known method is that the necessary measuring time is on the order of several minutes, in order to achieve sufficient accuracy by averaging over a great number of measurements and subsequent postprocessing of the data. The reasons for this are, first of all, the optical conditions in the skin.

Secondly, the measuring conditions on the living object influence the results of the measurement.

An object of the present invention is to provide the fastest possible and at the same time reliable measuring instrument and measuring method, respectively, for determining a property of the skin structure, particularly of the tissue fluid.

### SUMMARY OF THE INVENTION

This objective is achieved by the features according to the present invention. In this context, to scatter back a portion of the object beam directed into the skin, it is provided to introduce into the skin an implant having a front side facing the incident object beam and a back side facing away from it, and having a refractive index different from the surrounding skin substance.

The implant makes it possible to take optical measurements at the defined transition between the skin substance, thus in particular the tissue fluid, and the implant. In this manner, relatively precise and reliable measuring results are obtained. Various alternative forms of the measuring device or the procedure in the case of the method are obtained in that, to effectuate a depth scan in the skin and thereby to produce the intensity modulation, a scanning device is provided which is set up in such a way that the optical path length of the reference beam in the reference branch or the wavelength of the light source is altered in a defined manner, or that a spectral resolution and evaluation are carried out in the detector device, in the course of which, a conversion into the local space is performed.

The following measures contribute to the accuracy and reliability of the measurement: The implant is provided with a plurality of boundary surfaces relative to the tissue fluid between its front side and its back side, and the evaluation is planned in such a way that, with respect to the passage of the object beam through the implant, the depth-dependent decrease of the scattered light and/or the average optical path length is/are ascertained on the basis of the intensity modulation in the interference signal, accompanied by evaluation of an average refractive index for determining a property of the skin structure.

The measuring conditions may be optimized by forming, structuring or coating the implant in such a way that the front-side signal coming from its front side and the back-side signal coming from its back side are especially prominent.

In order to suppress disturbances resulting from a dispersion of the dermal layers to the greatest extent possible, a further advantageous measure is to introduce in the reference branch, a dispersion-compensating medium matched to the passage section of the skin.

In the case of the method, an advantageous procedure with respect to the evaluation is that, to determine the property of the skin structures on the basis of the intensity modulation, the depth-dependent decrease of the scattered light and/or the average optical path length within the implant is/are ascertained from an evaluation of an average refractive index, in doing so, the geometric wavelength being known.

An advantageous practical application of the device and the method is to utilize them to determine the blood-sugar concentration, in doing so, use being made of the relationship, known per se, between the sugar concentration in the tissue fluid and the blood.

### BRIEF DESCRIPTION OF THE DRAWING

The FIGURE shows an interferometric measuring instrument according to the present invention.

### DETAILED DESCRIPTION

The FIGURE shows a schematic representation of an interferometric measuring instrument having an interferometer part featuring an object branch 13 in which the skin section to be measured is introduced, and a reference branch 14 having a reference 16. An input beam 11 is guided from a light source 10 to a beam splitter 12, which splits it into an object beam 13.1 guided via object branch 13, and a reference beam 14.1 guided via reference branch 14. The object beam scattered back from skin 15 and the reference beam scattered back from reference 16 are superimposed and brought to interference, for which the optical path length of reference branch 14 and of object branch 13 are adapted accordingly. The interfering radiation is picked up by an image recorder of a detector device 18, and the intensity modulations resulting due to the interference are evaluated in an adjacent evaluator. A dispersion-compensating medium 19 is introduced into reference branch 14 to compensate for the dispersion in the dermal layers.

The interferometric measuring instrument is used for the purpose of ascertaining depth-resolved optical information, particularly the intensity scattered back at a specific depth of the skin structure. Depending on the type of interferometric set-up, light source 10 is either a “white-light source” with which a specific coherence length is associated, or a narrow-band, tunable (laser) light source.

The light in object branch 13 interacts with the substances located there, an implant being introduced as an exceptional feature into the skin in order to form one or more defined boundary surfaces. A light fraction is returned in the direction of the beam splitter as a function of the difference in refractive index between the skin substance, particularly the tissue fluid, adjacent to the boundary surfaces of the substrate, and the implant material.

The light in reference branch 14 strikes reflecting reference 16, e.g., a plane mirror, and is reflected back from there. Depending on the amount of light returned in object branch 13, it may also be advantageous to suitably balance the reflectance in reference branch 14 by further measures. For example, for the depth scanning of the skin section to be measured, reference 16 in reference branch 14 may be moved in the direction of the beam splitter.

Depending on the interferometric set-up or method, the depth scan is carried out by moving reference 16 in reference branch 14 or by altering the wavelength of light source 10 in defined manner. In a further alternative, detector device 18 is designed for the spectral dispersion of the detected radiation, and the evaluation is based on the spectrally resolved radiation, a conversion into the local space being carried out according to methods known per se.

Recombined and interfering radiation 17 returning from object branch 13 and reference branch 14 produces in detector device 18 a signal proportional to the instantaneous light intensity, corresponding to the intensity modulation, the interferometric intensity modulation being carried out as a function of depth by the scanning mechanism indicated above. The magnitude of the intensity modulation at a specific scanning position gives information about the amount of back-scattered light in the object branch at a specific depth.

Implant 20 is introduced into the skin in order to take the non-invasive optical measurements then following, a biocompatible implant being used. Given a known and fixed refractive index of implant 20, the amount of back-scattered light at the respective boundary surface between implant 20 and tissue fluid is now determined based on the difference in refractive index of the two media. If a property of the skin substance determining the refractive index of the skin substance in question is known, it is therefore possible to draw conclusions with respect to this property by way of the difference in refractive index ascertained. For example, in this way it is possible to ascertain the change in the refractive index of the tissue fluid caused by the blood-sugar concentration. When the measuring light passes through a multitude of boundary surfaces between the implant material and the surrounding skin substance, thus, especially tissue fluid, a depth-dependent decrease of the back-scattered light is measurable accordingly. In order to obtain a multitude of boundary surfaces, advantageously a microstructuring of implant 20 with many channels or cavities, or a porous material is used. The depth-dependent decrease of scattered light observable in such an implant 20 may thus be used for measuring the change in refractive index, and therefore, for example, for determining blood sugar.

For the case of a microstructured or porous implant 20, a further method is obtained for measuring the change in refractive index. In the passage through implant 20, the light experiences an average refractive index that is yielded from the averaging of the optical path lengths in the implant medium and in the skin substance, especially tissue fluid. This average optical path length is determined in the optical depth scan, when the front side and back side of the implant are derived from the measuring signal. The measured difference between the two points of the front side and the back side yields the optical path length in implant 20. In this context, implant 20 may also be formed, structured or coated in such a way that the front-side signal and back-side signal are especially prominent in the measuring signal. For example, one possibility here is the coating with a medium having a particularly high optical refractive index.

On the other hand, since, in contrast to the optical path length, the geometrical path length remains constant, a change in the measured optical path length may be attributed to a change in the refractive index of the skin substance or tissue fluid within implant 20. In turn, this is in direct relation to the property to be determined, thus, for example, the blood-sugar concentration. Therefore, a second physical measured quantity is available in the same measurement, which yields further information, an increase in accuracy or a verification of the measuring result alternatively or in addition to the first-named method of the depth-dependent change in scattered light.

With implant 20, a defined fixed quantity is predefined for the optical measurement, thereby promoting the measuring accuracy and reliability of the measurement result, as well as the measuring speed considerably.

1-8. (canceled)

9. An interferometric measuring instrument for taking optical measurements on skin structures of skin, comprising:
a beam splitter for splitting an input beam, emitted by a light source, into an object beam and a reference beam;
an object branch via which the object beam is directed to the skin, and the object beam, scattered back from the skin structures, is returned;
a reference branch via which the reference beam is directed to a reference and returned from the reference; and
a detector device for picking up recombined and interfering radiation of the back-scattered object beam and of the returned reference beam and for evaluating an intensity modulation obtained due to the interference; and
an implant introduced into the skin for scattering back a portion of the object beam directed into the skin, the implant having a front side facing the incident object beam and a back side facing away from the incident object beam, the implant having a refractive index different from a surrounding skin substance.

10. The measuring instrument according to claim 9, further comprising a scanning device for effectuating a depth scan in the skin and thereby for producing the intensity modulation, the scanning device being set up in such a way that an optical path length of the reference beam in the reference branch or a wavelength of the light source is altered in a predefined manner, or that a spectral resolution and evaluation are carried out in the detector device, in the course of which, a conversion into a local space is performed.

11. The measuring instrument according to claim 9, wherein the implant has a multitude of boundary surfaces relative to a tissue fluid between its front side and its back side, and the evaluation is planned in such a way that, with respect to the passage of the object beam through the implant, at least one of (a) a depth-dependent decrease of the scattered light and (b) an average optical path length is ascertained on the basis of an intensity modulation in an interference signal, accompanied by evaluation of an average refractive index for determining a property of the skin structure.

12. The measuring instrument according to claim 9, wherein the implant is formed, structured or coated in such a way that a front-side signal coming from its front side and a back-side signal coming from its back side are prominent.

13. The measuring instrument according to claim 9, further comprising a dispersion-compensating medium matched to a passage section of the skin and introduced into the reference branch.

14. The measuring instrument according to claim 9, wherein the optical measurements are for determining a blood-sugar concentration.

15. An interferometric method for taking optical measurements on skin structures of skin, comprising:
splitting an input beam coming from a light source into a reference beam guided to a reference and an object beam guided to the skin;
bringing to interference the reference beam and the object beam scattered back from the reference and from the skin;
detecting and evaluating an intensity modulation obtained due to the interference for determining a property of the skin structures;
introducing an implant that has a refractive index different from a surrounding skin substance into the skin; and
evaluating the intensity modulation brought about by light scattered back at at least one boundary surface of the implant relative to tissue fluid for determining the property of the skin structures.

16. The method according to claim 14, further comprising, to determine the property of the skin structures on the basis of the intensity modulation, ascertaining at least one of (a) a depth-dependent decrease of the scattered light and (b) an average optical path length within the implant, from an evaluation of an average refractive index, in doing so, a geometric wavelength being known.

17. The method according to claim 15, wherein the optical measurements are for determining a blood-sugar concentration.
